(19) United States
(12) Patent Application Publication
(10) Pub. No.: US 2013/0034868 A1
(43) Pub. Date: Feb. 7, 2013

(54) METHOD AND USE FOR ASSESSING RADIATION THERAPY IN ACCORDANCE WITH SAA

(71) Applicant: Pei-Ru LIAO, Taipei City (TW)

(72) Inventor: Pei-Ru LIAO, Taipei City (TW)

(21) Appl. No.: 13/566,782

(22) Filed: Aug. 3, 2012

Related U.S. Application Data
(60) Provisional application No. 61/514,970, filed on Aug. 4, 2011.

(51) Int. Cl. G01N 33/566

(52) U.S. Cl. 435/7.92

(57) ABSTRACT

A method for assessing a subject to receive radiation therapy in accordance with the present invention includes the steps of obtaining a body fluid sample from the subject, measuring the concentration of serum amyloid A proteins of the body fluid sample and comparing the concentration of the serum amyloid A proteins with a range of values to determine whether the subject is suitable to receive radiation therapy. Additionally, the present invention also provides a use of serum amyloid A proteins for assessing whether a subject is suitable to receive radiation therapy.

### CROSS REFERENCE TO RELATED APPLICATIONS

The non-provisional patent application claims priority to U.S. provisional patent application with Ser. No. 61/514,970 filed on Aug. 4, 2011. This and all other extrinsic materials discussed herein are incorporated by reference in their entirety.

### BACKGROUND OF THE INVENTION

1. Field of Invention

The present invention relates to a method for assessing whether a subject can receive radiation therapy and a use of serum amyloid A proteins.

2. Related Art

Cancer and malignant tumor are kinds of disease resulting from uncontrolled cell proliferation such that they are almost impossible to prevent and cure. Except for the issue of uncontrolled cell proliferation, cancer metastasis often occurs and cancer cells spread from a primary site to other important tissues or organs during the final stage of the cancer. Both of them are critical influences to the normal physical operation of human body with an extremely high mortality rate.

Presently, there are three kinds of methods for treating cancer: surgery, chemotherapy and radiation therapy, wherein the radiation therapy plays a fairly important role in the field of current cancer treatment.

Radiation therapy can be widely applied to most cancers or malignant tumors. More importantly, radiation therapy contributes an excellent therapy effect in both of independent use and combination use with other treatments such as surgery or chemical therapy. In clinic, the proper dose of radiation therapy can be estimated in accordance with the stages of cancer and the severity of metastasis, patient's physiological status or combination with other treatments in order to effectively destroy cancer cells or inhibit the proliferation of tumor cells with radiation. Radiation therapy has a feature of local treatment so as to prevent undesired side effects of, for example, effecting the growth of normal cells and inducing uncomfortable feeling which patients take chemical medicines often have. Additionally, radiation therapy also has an advantage of a better prognosis as well.

However, it is known that patients previously receiving radiation therapy for lung cancer or malignant tumors occurring in chest such as mammary adenocarcinoma, esophageal cancer or malignant lymphoma are at an extremely high rate to suffer from radiation pneumonitis (RP). The symptoms of RP includes for example extensive pulmonary fibrosis, pulmonary vasoconstriction, thickened inner membrane in capillaries, sclerosis, high resistance in pulmonary circulation and high pressure in pulmonary artery caused by coarctation or blocking of blood vessels, etc. Moreover, with the progression disease of pulmonary fibrosis, it is often to induce infection in respiratory tract and thereby deteriorate other symptoms of respiratory tracts.

According to previous studies, RP can be suppressed or prevented by adjusting highly related factors such as radiation dose and irradiation range. However, those means of adjustment are only considered after patients have been observed with the symptoms of RR. Therefore, they are still not effective solutions for preventing the occurrence of RP even with a compromise of reducing radiation dose to sacrifice therapeutic effect. In addition, because RP has already been induced in patients, those means of adjusting dose or irradiation range are only used as a remedy to reduce the damage caused by RP rather than a proper pre-assessment method.

### SUMMARY OF THE INVENTION

In view of the foregoing, the purpose of the present invention is to provide a method and a use of serum amyloid A proteins (SAAs) for assessing whether a subject can receive radiation therapy. The result of the method can be considered as an assessment indicator to prevent an issue that patients suffer from RP after receiving radiation therapy.

To achieve the purpose as described above, the present invention provides a method for assessing a subject to receive radiation therapy includes obtaining a body fluid sample from the subject; measuring the concentration of serum amyloid A proteins of the body fluid sample; and comparing the concentration of the serum amyloid A proteins with a range of values to determine whether the subject can receive radiation therapy.

In one embodiment of the present invention, the body fluid sample includes blood, serum, plasma, tissue fluid, lymph, urine, or saliva sample.

In one embodiment of the present invention, the method is applied to assess whether the subject can receive radiation therapy for cancer treatment.

In one embodiment of the present invention, the cancer includes lung cancer, mammary adenocarcinoma, esophageal cancer, or malignant lymphoma.

In one embodiment of the present invention, the range of values is between about 0.05 μg/ml to about 5,000 μg/ml, preferably, between about 50 μg/ml to about 200 μg/ml and, more preferably, about 100 μg/ml.

In one embodiment of the present invention, the serum amyloid A proteins include serum amyloid A1 protein, serum amyloid A2 protein, serum amyloid A3 protein, serum amyloid A4 protein, acute serum amyloid A protein, constitutive serum amyloid A protein or the combination thereof.

In one embodiment of the present invention, the concentration of serum amyloid A proteins of the body fluid sample is measured by photometry or protein chip assay. Preferably, the measurement is achieved by enzyme-linked immunosorbent assay (ELISA).

In one embodiment of the present invention, the comparison of the concentration of the serum amyloid A proteins with the range of values is to determine that the subject can receive the radiation therapy while the concentration of the serum amyloid A proteins is lower than the range of values or the subject can not receive the radiation therapy while the concentration of the serum amyloid A proteins is higher than or falls into the range of values.

In addition, the present invention also provides a use of serum amyloid A proteins for assessing whether a subject can receive radiation therapy.

In one embodiment of the present invention, whether the subject can receive the radiation therapy is assessed by measuring the concentration of serum amyloid A proteins in a body fluid sample of the subject is higher than a range of values.

In summary, a method for assessing a subject to receive radiation therapy and a use of serum amyloid A proteins in accordance with the present invention can reduce the risk of RP occurrence and assess the potential severity of the symptoms of RP by measuring the concentration of serum amyloid A proteins (SAAs) of a body fluid sample of a subject and then comparing the concentration of SAAs with a range of values to determine whether the subject can receive radiation therapy before radiation therapy is administrated. In practice, the present invention has advantages of preventing an issue that a subject treated with radiation therapy is often induced with RP by measuring the concentration of SAAs of a body fluid sample of the subject having tumors or cancer as an assessment indicator.

In detail, while the concentration of SAAs of a body fluid sample of a subject is lower than a range of values, it indicates that RP will not be induced in the subject or only mild RP will be induced after the treatment of radiation therapy. However, while the concentration of SAAs of the body fluid sample of the subject is higher than or falls into the range of values, it indicates that RP, even severe RP, will most likely be induced in the subject after the treatment of radiation therapy.

According to the research results of the present inventors, that the concentration of SAAs of body fluid samples has a higher positive correlation with the rate of RP occurrence and the severity degrees of RP comparing to other related inflammatory factors in prior art such as interleukin-6 (IL-6) or C-reactive protein (CRP) contributes SAAs as a proper assessment indicator. Since the use of the concentration of SAAs to assess whether a subject can receive radiation therapy has never been disclosed in prior art and, surprisingly, the concentration of SAAs was always considered being less relative to the inflammatory status of a subject than those aforementioned related inflammatory factors in previous studies, the inventors of the present invention provide significant contributions. Moreover, because SAAs are not trace molecules in body fluids, the concentration of SAAs has advantages for easy measurement and quantitative analysis and can be wildly applied in many fields.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a flow chart of a method for assessing a subject to receive radiation therapy in accordance with the present invention;

FIG. 2A to FIG. 2D shows a data of the correlation of the concentrations of SAA, CRP, IL-6 and MLD with mild RP and severe RP;

FIG. 3A shows a data of the sensitivity and specificity of the concentrations of SAA, CRP, IL-6 and MLD in assessing mild RP and severe RP;

FIG. 3B shows a data of the sensitivity and specificity of SAA and MLD with different irradiation doses in assessing mild RP and severe RP;

FIG. 4A shows a statistic data of the overall survival of the patients with mild RP and severe RP; and

FIG. 4B shows a statistic data of the overall survival of the patients with high concentration of SAA and low concentration of SAA.

### DETAILED DESCRIPTION OF THE INVENTION

A method for assessing a subject to receive radiation therapy and a use of SAAs for assessing whether a subject can receive radiation therapy will be apparent from the following detailed description, which proceeds with reference to the accompanying drawings. The assessment result can be considered as an indicator to consider whether the subject is suitable to receive radiation therapy or plan how radiation therapy can be administrated to the subject for treating cancer or malignant tumor so as to prevent the induction of RP effectively.

It is known that after a patient with lung cancer, mammary adenocarcinoma, esophageal cancer, malignant lymphoma or other malignant tumors in chest is treated with radiation therapy, normal lung tissues or cells in the patient's body will likely be injured to induce RP. It is also known that a patient in inflammation have a fairly high risk of RP induction after receiving radiation therapy comparing a patient without inflammation. Those observations indicate that RP occurrence is highly related to inflammation in the patient before radiation therapy. However, the model for assess the risk has not been established yet.

Therefore, the inventors of the present invention are diligent to find an assessment indicator and a method for assessing and predicting whether RP will be induced after a subject receives radiation therapy. The inventors discovered proteins, which have a signal between 2,000˜20,000 m/z and a size of 11,480 Da, in body fluid samples. According to pre-treatment measurement data, the inventors discovered that the intensity of the protein signal of subjects, who would not suffer from RP or only suffer from mild RP after receiving radiation therapy, was significantly weaker than that of subjects, who would suffer from moderate or severe RP after treatment of radiation therapy, in pre-treatment measurement. The result was further confirmed by that the greatest average intensity of the protein signal was measured in subjects, who would suffer from severe RP after receiving radiation therapy. It suggests that this protein, as an assessment indicator, is capable of determining the rate of a subject to suffer from mild RP or severe RP after treatment.

The proteins mentioned above are further identified as serum amyloid A proteins (SAAs), which are proteins involving in inflammatory responses.

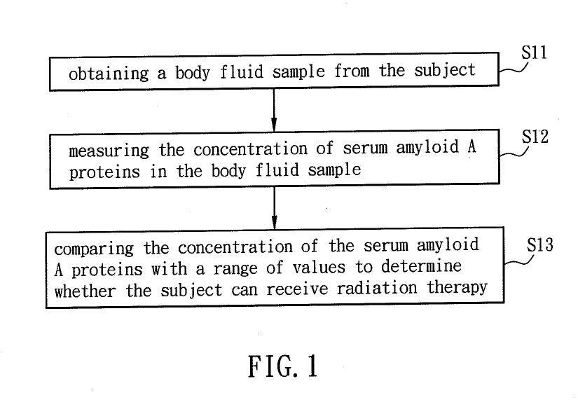

According to the aforementioned research result, the present invention discloses a method for assessing a subject to receive radiation therapy. As shown in FIG. 1, the method includes the steps of obtaining a body fluid sample of the subject (step S11); measuring the concentration of serum amyloid A proteins (SAAs) of the body fluid sample (step S12); and comparing the concentration of the SAAs with a range of values to determine whether the subject can receive radiation therapy (step S13).

The term “subject” described herein refers to an organism having benign or malignant tumors or cancer. Preferably, the site of benign or malignant tumors is in nasal or thoracic cavity. In one embodiment of the present invention, the cancers include nasopharyngeal cancer, lung cancer, esophageal cancer, breast cancer, etc. The organism substantially refers to mammals such as human, mouse, canine, feline, monkey, bovine, swine, caprine, etc. More preferably, the organism refers to human. In addition, the term “serum amyloid A proteins (SAAs)” described herein includes any kind of SAA isoforms, for example but not limited to serum amyloid A1 protein (SAA1), serum amyloid A2 protein (SAA2), serum amyloid A3 protein (SAA3), serum amyloid A4 protein (SAA4), acute serum amyloid A protein (A-SAA) and constitutive serum amyloid A protein (C-SAA).

FIG. 1 is a flow chart diagram of a method for assessing a subject to receive radiation therapy in accordance with a preferable embodiment of the present invention. The details of the method of the present invention will be clearly described in the following with FIG. 1.

The step S11 shown in FIG. 1 is obtaining a body fluid sample of the subject. The term “body fluid sample” refers to a portion separated or collected from any body fluid of a subject, such as blood, serum, plasma, tissue fluid, lymph, urine, saliva, etc. Certainly, the body fluid sample can include a sample which is treated by additional process to maintain the quality for measurement, or treated by other process such as centrifuging, purifying, condensing, freezing and/or preserving for subsequent measurement or process. In the present embodiment, the body fluid sample is preferably a serum, which is obtained by collecting a needed volume of blood from a subject and then centrifuging it to remove blood cells. In addition, sites for collecting the body fluid sample can be in, for example, vein, artery, lymph gland, lymphatic organ, peritoneal, vertebra, cerebrum or urinary bladder, or tumor or cancer sites or adjacent to tumor or cancer sites. Means of collecting the body fluid sample can be selected in accordance with sites for collecting the body fluid sample. Moreover, the body fluid sample can be collected for example but not limited to by direct extraction from the body of the subject or by separation from the body fluid conducted or excreted out of the body.

In the step S12, the measurement of the concentration of SAAs of the body fluid sample can be achieved by photometry. Practically, photometry includes enzyme-linked immunosorbent assay (ELISA). In more detail, methods for measurement can be in principle classified by measurement of the amount of total proteins or measurement of the amount of a specific protein in the body fluid sample. ELISA is one of the methods for measuring the amount of a specific protein.

If the method is selected to measure the amount of total proteins in the body fluid sample, the body fluid sample has to purify to isolate all of SAAs in the body fluid sample first and then proceed to measure the amount of SAAs. On the contrary, if the selected method can specifically measure the amount of SAAs in the body fluid sample, the body fluid sample needs no purification but is measured with specific reagents such as antibodies. The ELISA described herein includes for example but not limited to indirect ELISA, competitive ELISA and sandwich ELISA. In the present embodiment, the amount of total SAAs of the body fluid sample is preferably measured by ELISA.

However, the present invention can also be practiced with protein chips, including a protein chip analyzed by immunological assay, as assay platforms.

In photometry, the concentration of SAAs of the body fluid sample can be measured by detecting the intensity of a color signal generated after adding a proper reagent to induce oxidation-reduction reaction. For example, in the method selected to specifically measure the amount of SAAs, the types of substances coupled to antibodies decide the types or the wavelengths of generated signals. The substances can include enzymes, substrates for enzymes or chromogenic reagents. The enzymes including for example luceiferase, β-galactosidase, horseradish peroxidase and alkaline phosphatase are able to release detectable optical signals after reacting with proper substrates. Some other substrates are able to release detectable optical signals after reacting with proper enzymes. The chromogenic reagents including for example fluorescein isothiocynate, rhodamine, phycoerythrin fluorescent proteins, etc. are able to release detectable optical signals alone without reacting with any other substances.

No matter what the type of measurement methods is selected, the concentration of SAAs of the body fluid sample can be estimated by comparing the measured concentration of SAAs of the body fluid sample with that of a standard sample or the measured intensity of the generated optical signals with a reference value and calculated by statistics software. The calculation is well known to the person who skilled in the art such that the detail description will be omitted herein. However, the step S12 of measuring the concentration of SAAs of the body fluid sample can also be achieved by other means of assay such as Western blot, dot blot, etc. Additionally, the assay can be individually performed on a well plate, test strip or gel. The assay can also be achieved by combing a serious of analyses.

The step S13 is to compare the concentration of SAAs with a range of values to determine whether the subject can receive radiation therapy. The term “range of values” described herein mainly refers to a range covering a lower limit value through a upper limit value. In more detail, the range of values consists of the upper limit value, the lower limit value and every integer and integer with a decimal between the lower limit value through the upper limit value. According to the research results of the inventors, the concentration of SAAs of the body fluid sample of a subject is highly correlated with the rate that the subject suffers from RP after receiving radiation therapy. Meanwhile, SAAs are products produced in inflammation. Therefore, if the concentration of SAAs of the body fluid sample of a subject exceeds a range of values, it indicates that the subject is in a severe inflammation and has an extremely high rate to suffer from RP after receiving radiation therapy such that he/she is not suitable for radiation therapy in the moment. In contrast, if the concentration of SAAs of the body fluid sample of a subject is lower than a range of values, it indicates that the subject is in a mild inflammation or is not in inflammation. It also means that the subject has a low rate to suffer from RP after receiving radiation therapy such that the subject is suitable for radiation therapy. In brief, that determination of whether a subject can receives radiation therapy is decided by the concentration of SAAs of the body fluid sample of the subject. If the concentration is lower than the range of values, the subject can receive radiation therapy. On the contrary, if the concentration is higher than the range of values or falls into the range of values, the subject can't receive radiation therapy.

The time interval from the measurement of the concentration of SAAs of the body fluid sample of a subject to the first treatment of radiation therapy can be several hours to several days, preferably, 12 hours to 72 hours and, more preferably, 24 hours to 48 hours. In other words, the present invention can be practiced to measure the concentration of SAAs of the body fluid sample of a subject and then determine whether the subject can receive radiation therapy as plan 3 to 4 hours before the scheduled treatment of radiation therapy.

The range of values mentioned above can be between about 0.05 μg/ml to about 5,000 μg/ml, preferably, between about 50 μg/ml to about 200 μg/ml and, more preferably, 100 μg/ml. If the range of values is a single value, it indicates that the minimum and maximum of the range of values are extremely close such that the range of values only consists of the single value as a mean value and the single value with measurement errors. However, the range of values used herein is not be limited to the aforementioned but also can vary in accordance with different isoforms of SAAs, different means of measurement or different individuals for body fluid sample collection and different radiation dose administrating to the subject as long as corresponding to the concepts of the present invention.

To be noted, determination of whether a subject can receive radiation therapy is not only for identify subjects who have no risk to suffer from RP after radiation therapy but also for other even more important purpose such as assessment of rate or severity of RP induced by radiation therapy.

Specifically, RP can be graded with Grades 0˜4 according to National Cancer Institute Common Toxicity Criteria, Version 2.0. Grades 0˜2 RP are defined as mild RP and Grades 3˜4 RP are defined as severe RP. In the comparison of the concentration of SAAs of the body fluid samples of subjects with a range of values, it can identify the subjects who have an extremely high rate to suffer from RP, especially server Grade 3 or 4 RP, after receiving radiation therapy with the concentration is higher than the range of values or falls into the range of values. On the contrary, the subjects with the concentration lower than the range of values can be identified to suffer from no RP or just suffer from Grades 0˜2 RP after receiving radiation therapy.

Hence, the method of the present invention can not only predict whether a subject will suffer from RP due to the treatment of radiation therapy by but also assess the grades of induced RP by different ranges of values. It is convenient for clinical personnel to realize the potential risk before treatment. In the present embodiment, the range of values can be used to predict either Grades 0˜1 RP or Grades 2˜4 RP will be induced in patients. Certainly, in other embodiments, the other ranges of values can be used to predict either Grade 0 RP or Grades 1˜4 RP, Grades 0˜2 RP or Grades 3˜4 or Grades 0˜3 or Grade 4 will be induced in patients.

To improve the accuracy of the method of the present invention, the range of values can be not only a range covering lower limit value through upper limit value but also just a specific value. In practice, the range of value can be 100 μg/ml, which can be used as threshold to predict either mild RP or severe RP will be induced in a subject after the treatment of radiation therapy. Certainly, the range of values mentioned above includes a specific value and the specific value with its statistical significance. For example, a specific value plus or minus its standard deviation (SD) is considered as the specific value itself.

The following experiment demonstrates that the concentration of SAA can be an assessment indicator to predict the rate of a subject suffering from RP after receiving radiation therapy and/or the severity of the induced RP by selecting a specific concentration value as a range of values and proceeding the comparison of it with different concentrations of SAAs of body fluid samples collected from different tested subjects. The prediction has been further confirmed by diagnosing the physical condition of the subjects after radiation therapy. Therefore, the following experiment can clearly demonstrate a use of SAAs for assessing a subject to receive radiation therapy. The detailed descriptions for practice can refer to the content mentioned above.

The following experiment demonstrates that the concentration of SAAs of the present invention can be an assessment indicator to determine whether a subject can receive radiation therapy and effectively predict that the subject may suffer from mild RP or severe RP after receiving radiation therapy.

### EXPERIMENT

### The Concentration of Saa can be Used to Assess Whether a Subject can Receive Radiation therapy

Patients and Sera

There are 80 sera samples collected from lung cancer patients who do not receive radiation therapy yet. The 80 sera samples are immediately stored at 4° C., then quickly centrifuged at 1,000×g for 10 minutes, and then stored at −80° C.

Radiation Therapy

All patients are immobilized in the supine position to have computed tomography (CT) for treatment planning. A diaphragm compressor is used to restrict the amplitude of respiration of the patients to less than 15 mm. CT is performed with 5-mm-thick slice increments, and images are transferred to a Pinnacle treatment planning system. The simulated images are fused with the latest lung CT scans for contouring. In principle, at least 50 Gy are delivered with the conventional fractionation schedule. The clinical target volume (CTV) covers the gross tumor volume (GTV) plus an expansion of 10-20 mm. The CTV receives a dose of 40 Gy in 20 fractions, and the GTV receives a boost of another 10 to 30 Gy in 5-15 fractions. Concomitant chemotherapy is allowed to treat the patients. Dose Volume Histograms (DVH) are generated. Spinal cord tolerance dose is set on 4,500 cGy, and the upper limit of GTV dose is generally set on a mean lung dose (MLD) of less than 1700 cGy. Because an ipsilateral lung inflammatory condition cannot be ameliorated by the contralateral lung, the MLD is calculated from the major irradiated single lung volume, excluding the GTV, instead of using paired lung calculations.

After receiving radiation therapy, the follow-up visits are arranged monthly for the first 3 months and are subsequently scheduled every 2 months for the first year to evaluate treatment response, toxicities and diseases status. RP is graded based on National Cancer Institute Common Toxicity Criteria, Version 2.0 as Grades 0˜4. Grade 0 stands for no symptoms of RP; Grades 1˜2 are defined as mild RP; and Grades 3˜4 are defined as severe RR. The grades of RP are calculated after the patients are treated with radiation therapy.

Measurement of the Related Inflammatory Factors

There are 3 inflammatory factors: SAAs, IL-6 and C-reactive protein (CRP) to be measured in the present experiment. IL-6 and CRP are control groups relative to SAAs. The concentrations of SAAs and IL-6 in sera of patients are using ELISA kit manufactured by Invitrogen (Camarillo, Calif., USA) and R&D systems (Minneapolis, Minn., USA), respectively, according to the manufacturers' instructions. All ELISAs are performed in duplicate. The protocol and the materials used therein of the ELISA and immunoassay should be understood by the person who skilled in the art such that they are omitted herein.

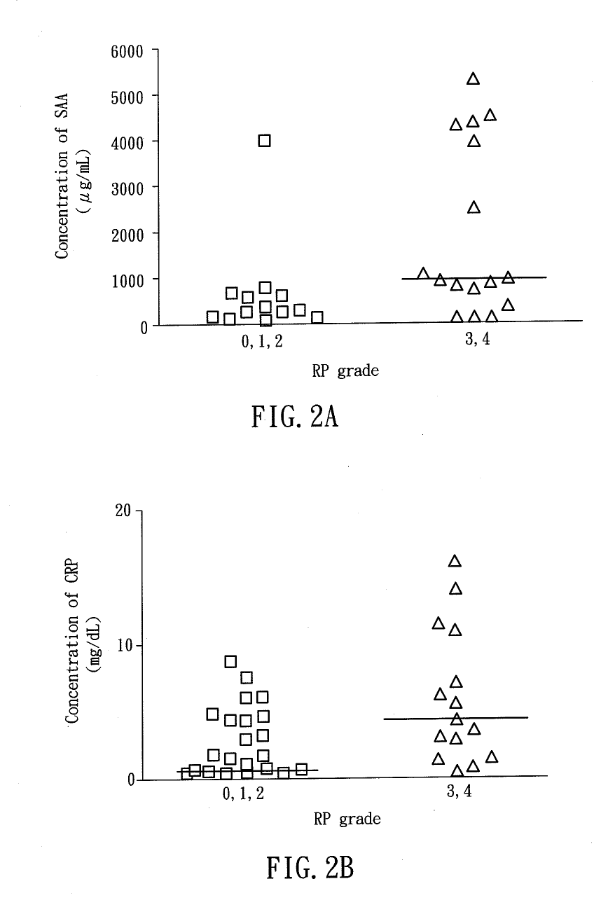

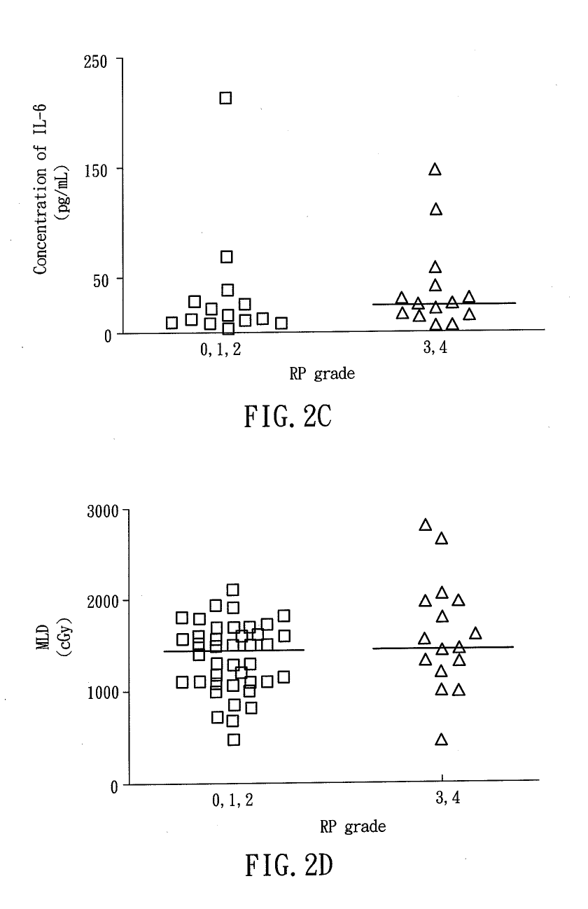

The sera samples from 58 patients are measured and graded into Grades 0˜4 according to the severity of induced RP after the patients receive radiation therapy. FIGS. 2A to FIG. 2D show the statistic data of the correlations of the measured concentrations of SAAs, CRP, IL-6 and MLD and the RP grades after patients receive radiation therapy. As shown in FIG. 2A, the statistic medians of the concentration of SAAs of the patients with severe RP and mild RP are 935 μg/mL and 32 μg/mL (P<0.0001), respectively; as shown in FIG. 2B, the statistic medians of the concentration of CRP of the patients with severe RP and mild RP are 4.39 mg/mL and 0.75 mg/mL (P<0.0001), respectively; and as shown in FIG. 2C and FIG. 2D, because the P values of the concentration of IL-6 and MLD are P=0.04 and P=0.333, respectively, both of them have no significant differences in statistics.

The results described above demonstrate that the concentrations of SAAs and CRP of the patients that have mild RP and severe RP after receiving the radiation therapy have more significant difference than the concentration of IL-6 and MLD, and especially, the concentration of SAAshas more significant difference than the concentration of CRP. Hence, it suggests that the concentration of SAAscan be used to predict the grades of induced RP as assessment indicator after subjects receive radiation therapy.

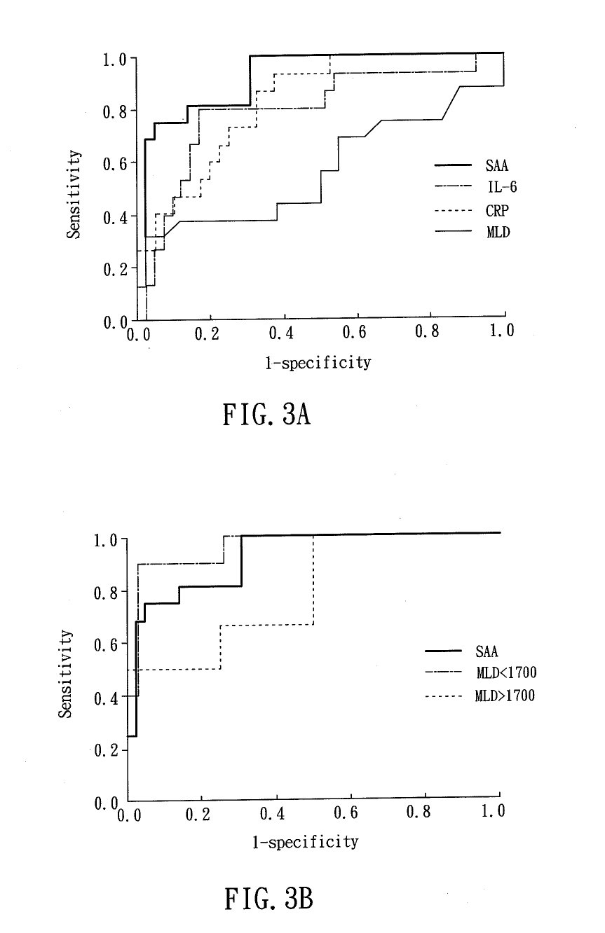

Besides, the present experiment also demonstrates that the correlation of the sensitivity and the specificity of the subjects with mild RP or with severe RP with the four groups: SAAs, IL-6, CRP and MLD. The results are as shown in FIG. 3A and FIG. 3B, the area under curves (AUC) are quantified so as to compare the sensitivity and the specificity of the different factors, the concentrations of SAAs, CRP, IL-6 and MLD, in assessing RP. As shown in FIG. 3A, the AUG of the concentrations of SAAs, CRP, IL-6 and MLD are 0.920 (0.847˜0.993 is within the 95% confidence interval (CI)), 0.827 (0.717˜0.920 fall within the 95% confidence interval (CI)), 0.792 (0.648˜0.935 fall within the 95% confidence interval (CI)) and 0.562 (0.374˜0.751 fall within the 95% confidence interval (CI)), respectively. The AUG of the concentration of SAAs is apparently higher than those of CRP, IL-6 and MLD, and it suggests that the concentration of SAAs has better sensitivity and specificity to assess that a subject suffers RP after receiving radiation therapy. In addition, as shown in FIG. 3B, the concentration of SAAs also has better sensitivity and specificity than MLD>1,700 cGy and MLD<1,700 cGy in assessing RP. The sensitivity and specificity of combined SAAs and MLD in assessing RP are 90.0% and 97.1%, respectively.

Predicting the overall survival of the patients after receiving radiation therapy by the concentration of SAAs.

Overall survival (OS) is calculated from the date of 3D-CRT administration to the date of death of patients. OS is estimated as a function of time by the Kapla-Meier method. SPSS software version 13.0 (SPSS Inc., Chicago, Ill., USA) and GraphPad Prism 5.0 (GraphPad Software Inc., San Diego, Calif.) are used for statistical analysis.

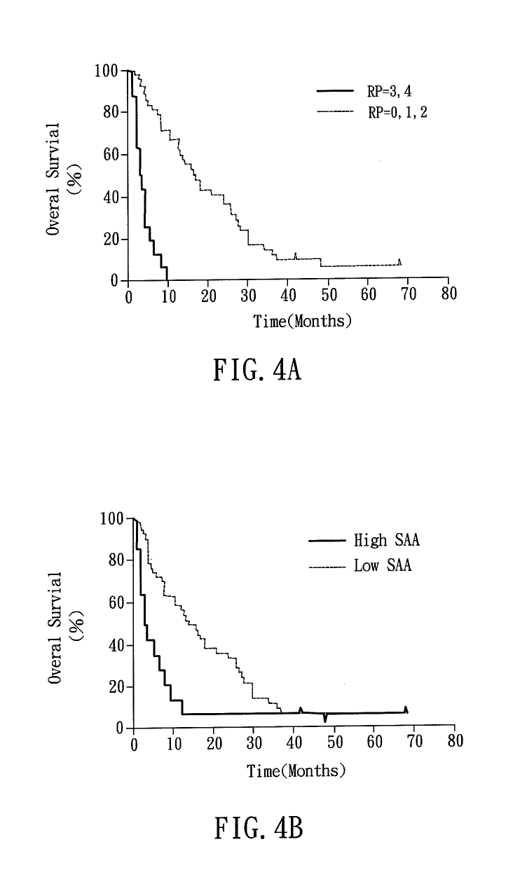

As shown in FIG. 4A, patients with severe RP have a significantly poorer survival rate than patients with mild RP (P<0.001) in accordance with the statistics data. As shown in FIG. 4B, patients with high concentration of SAAs (670 μg/mL) in sera also have poorer survival rate than patients with low concentration of SAAs in sera. According to FIG. 4A and FIG. 4B, both of the patients who have severe RP after receiving radiation therapy and the patients who have high concentrations of SAAs before receiving radiation therapy have poor survival rate. It suggests that the concentration of SAAs and the severity of RP is highly correlated, and it further suggests that the concentration of SAAs can be an assessment indicator to assess whether a subject suffers from RP after receiving radiation therapy.

In summary, a method for assessing a subject to receive radiation therapy and an use of serum amyloid A proteins in accordance with the present invention can decrease the possibility of RP occurrence and assess the potential severity of RP symptoms by measuring the concentration of serum amyloid A proteins of the body fluid sample of a subject and then comparing the concentration of the serum amyloid A proteins with a range of values to determine whether the subject is suitable to receive radiation therapy before radiation therapy is given. In practice, the present invention has advantages to preventing the issues that the subject received radiation therapy is often induced with RP by measuring the concentration of serum amyloid A proteins in the body fluid sample of the subject having tumor or cancer to be as an assessing indicator.

In detail, while the concentration of SAAs of a body fluid sample of a patient is lower than a range of values, it is belief that RP will not be induced in the subject or only mild RP will be induced after radiation therapy is used on the subject. However, while the concentration of SAAs of the body fluid sample of a patient is higher than or falls into the range of values, it is belief that RP, even severe RP, will be induced in the subject with extremely high possibility after radiation therapy is used on the subject.

According to inventors' research, that the concentration of SAAs of the body fluid sample has a higher positive correlation with the possibility of RP occurrence and the severity grades of RP comparing to other inflammatory factors in prior art such as interleukin-6 (IL-6) or C-reactive protein (CRP) contributes SAAs as a proper assessment indicator. Since the use of the concentration of SAAs to assess whether a subject is suitable to receive radiation therapy has never been disclosed in prior art and, surprisingly, the concentration of SAAs was considered being less relative to the inflammatory status of a subject than that of the aforementioned inflammatory factors in previous studies, the inventors of the present invention has significant contributions. Besides, because SAAs are not trace molecules existing in the body fluid, the concentration of SAAs has advantages of easy measurement and quantitative analysis and can be wildly applied in many fields.

What is claimed is:

1. A method for assessing a subject to receive radiation therapy comprising the steps of:
obtaining a body fluid sample from the subject;
measuring the concentration of serum amyloid A proteins of the body fluid sample; and
comparing the concentration of the serum amyloid A proteins with a range of values to determine whether the subject can receive radiation therapy.

2. The method of claim 1, wherein the body fluid sample includes blood, serum, plasma, tissue fluid, lymph, urine or saliva sample.

3. The method of claim 1 is applied to assess whether the subject can receive radiation therapy for cancer treatment.

4. The method of claim 3, wherein the cancer includes lung cancer, mammary adenocarcinoma, esophageal cancer, or malignant lymphoma.

5. The method of claim 1, wherein the range of values is between 0.05 μg/ml to 5,000 μg/ml.

6. The method of claim 1, wherein the range of values is between 50 μg/ml to 200 μg/ml.

7. The method of claim 1, wherein the serum amyloid A proteins includes serum amyloid A1 protein, serum amyloid A2 protein, serum amyloid A3 protein, serum amyloid A4 protein, acute serum amyloid A protein, constitutive serum amyloid A protein or the combination thereof.

8. The method of claim 1, wherein the concentration of serum amyloid A proteins of the body fluid sample is measured by photometry or protein chip assay.

9. The method of claim 1, wherein the comparison of the concentration of the serum amyloid A proteins with the range of values is to determine that the subject can receive the radiation therapy while the concentration of the serum amyloid A proteins is lower than the range of values or the subject can not receive the radiation therapy while the concentration of the serum amyloid A proteins is higher than or falls into the range of values.

10. A use of serum amyloid A proteins for assessing whether a subject can receive radiation therapy.

11. The use of claim 10, wherein whether the subject can receive the radiation therapy is assessed by measuring the concentration of serum amyloid A proteins in a body fluid sample of the subject is higher than a range of values.
